(19) United States
(12) Patent Application Publication
(10) Pub. No.: US 2025/0250317 A1
(43) Pub. Date: Aug. 7, 2025

(54) METHODS AND MATERIALS FOR TREATING GRAFT-VERSUS-HOST DISEASE

(71) Applicant: Mayo Foundation for Medical Education and Research, Rochester, MN (US)

(72) Inventors: Saad J. Kenderian, Rochester, MN (US); Mehrdad Hefazi Torghabeh, Rochester, MN (US)

(21) Appl. No.: 19/096,046

(22) Filed: Mar. 31, 2025

Related U.S. Application Data
(62) Division of application No. 17/045,372, filed on Oct. 5, 2020.
(60) Provisional application No. 62/655,013, filed on Apr. 9, 2018.

(51) Int. Cl. C07K 14/725; A61K 40/11; A61K 40/22; A61K 40/31; A61K 40/41; A61K 40/42; A61P 37/06; C07K 14/705; C07K 16/28

(52) U.S. Cl. CPC C07K 14/7051; A61K 40/11; A61K 40/22; A61K 40/31; A61K 40/418; A61K 40/4254; A61P 37/06; C07K 14/70517; C07K 14/70521; C07K 14/70532; C07K 16/2839; C07K 16/2896; C07K 2317/622; C07K 2319/02; C07K 2319/03

(57) ABSTRACT

This document provides methods and materials involved in treating a mammal (e.g., a human) having (or risk of developing) graft-versus-host disease (GVHD). For example, T cells (e.g., regulatory T cells) expressing one or more antigen receptors targeting one or more epithelial-specific antigens are provided. Also provided are methods for administering T-cells expressing one or more antigen receptors targeting one or more epithelial-specific antigens to a mammal having (or at risk of developing) GVHD to treat the GVHD.

### CROSS REFERENCE TO RELATED APPLICATIONS

This application is a National Stage application under 35 U.S.C. § 371 of International Application No. PCT/US2019/026544, having an International Filing Date of Apr. 9, 2019, which claims benefit of priority from U.S. Provisional Application Ser. No. 62/655,013, filed on Apr. 9, 2018. The disclosure of the prior application is considered part of (and is incorporated by reference in) the disclosure of this application.

### BACKGROUND

### 1. Technical Field

This document relates to methods and materials involved in treating a mammal (e.g., a human) having or at risk of developing graft-versus-host disease (GVHD). For example, one or more T cells (e.g., one or more regulatory T cells (Tregs)) expressing one or more antigen receptors targeting one or more epithelial-specific antigens can be administered to a mammal (e.g., a human) having GVHD (or at risk of developing GVHD) to treat the GVHD (or to reduce the risk of developing GVHD).

### 2. Background Information

Acute GVHD (aGVHD) is a major cause of morbidity and mortality after allogeneic hematopoietic cell transplantation (HCT) (Socie et al., Blood, 114 (20): 4327-36 (2007); and Martin et al., Bone Marrow Transplant, 21 (8): 1343-59 (2014)). There are currently no standard treatments beyond steroids; however, even with steroid treatment, mortality at 12 weeks for aGVHD patients is 63% (Yalniz et al., Biology of Blood and Marrow Transplant, 23 (9): 1478-1484 (2017)).

### SUMMARY

This document provides methods and materials involved in treating a mammal (e.g., a human) having or at risk of developing GVHD. For example, one or more T cells (e.g., one or more Tregs) expressing one or more antigen receptors targeting one or more epithelial-specific antigens can be administered (e.g., by adoptive transfer) to a mammal having GVHD (or at risk of developing GVHD) to treat the GVHD (or to reduce the risk of developing GVHD). In some cases, a chimeric antigen receptor (CAR) targeting an epithelial-specific antigen (e.g., epithelial cadherin (E-cadherin, also referred to as CDH1)) can be expressed by a Treg to target the Treg to epithelial tissues. In some cases, a cluster of differentiation 103 (CD103) polypeptide targeting an epithelial-specific antigen (e.g., CDH1) can be expressed on a Treg to target or redirect the Treg to epithelial tissues.

As demonstrated herein, donor-derived Tregs or autologous Tregs can be engineered to target host epithelial tissues by engineering the Tregs to express CARs targeting CDH1 (e.g., CDH1-CAR). Also as discussed herein, donor-derived Tregs or autologous Tregs can be engineered to target host epithelial tissues by engineering the Tregs to express a CD103 polypeptide. Tregs directed to host epithelial tissues can release inhibitory cytokines and reduce inflammation caused by graft versus host disease. Thus, Tregs directed to epithelial tissues can be incorporated into adoptive T cell therapies (e.g., CART cell therapies) to treat a mammal (e.g., a human) having GVHD (or at risk of developing GVHD) to treat the GVHD (or to reduce the risk of developing GVHD).

In general, one aspect of this document features a method for treating a mammal having GVHD. The method comprises (or consists essentially of or consists of) administering to the mammal a composition comprising regulatory T cells (Tregs) comprising exogenous nucleic acid encoding an antigen receptor targeting an epithelial-specific antigen, wherein the Tregs express the antigen receptor. The mammal can be a human. The GVHD can be acute GVHD or chronic GVHD. The GVHD can be GVHD that occurred following allogeneic transplantation. The epithelial-specific antigen can be E-cadherin (CDH1). The antigen receptor can be a chimeric antigen receptor. The chimeric antigen receptor can comprise a single chain variable fragment (scFv). The scFv can comprise a light chain and a heavy chain from an anti-CDH1 antibody. The anti-CDH1 antibody can be hSC10.17. The antigen receptor can comprise cluster of differentiation 103 (CD103). The Tregs, prior to the administration, can be engineered to express the antigen receptor ex vivo. A symptom of the GVHD can be reduced at least 10 percent.

In another aspect, this document features a method for treating a mammal at risk of developing GVHD. The method comprises (or consists essentially of or consists of) administering to the mammal a composition comprising regulatory T cells (Tregs) comprising exogenous nucleic acid encoding an antigen receptor targeting an epithelial-specific antigen, wherein the Tregs express the antigen receptor. The mammal can be a human. The GVHD can be acute GVHD or chronic GVHD. The mammal can be a mammal that received an allogeneic transplantation. The epithelial-specific antigen can be E-cadherin (CDH1). The antigen receptor can be a chimeric antigen receptor. The chimeric antigen receptor can comprise a scFv. The scFv can comprise a light chain and a heavy chain from an anti-CDH1 antibody. The anti-CDH1 antibody can be hSC10.17. The antigen receptor can comprise CD103. The Tregs, prior to the administration, can be engineered to express the antigen receptor ex vivo.

In another aspect, this document features a nucleic acid construct encoding a chimeric antigen receptor targeting an epithelial-specific antigen. The epithelial-specific antigen can be E-cadherin (CDH1). The chimeric antigen receptor can comprise a scFv. The scFv can comprise a light chain and a heavy chain from an anti-CDH1 antibody. The anti-CDH1 antibody can be hSC10.17. A heavy chain of the hSC10.17 can be encoded by a nucleic acid sequence set forth in SEQ ID NO:2. A light chain of the hSC10.17 can be encoded by a nucleic acid sequence set forth in SEQ ID NO:4. The chimeric antigen receptor can comprise a CD8 hinge region. The CD8 hinge region can be encoded by a nucleic acid sequence set forth in SEQ ID NO:5. The chimeric antigen receptor can comprise a CD28 transmembrane domain. The CD28 transmembrane domain can be encoded by a nucleic acid sequence set forth in SEQ ID NO:7. The chimeric antigen receptor can comprise a CD3zeta signaling domain. The CD3zeta signaling domain can be encoded by a nucleic acid sequence set forth in SEQ ID NO:8.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 contains a schematic of an exemplary method for treating aGVHD. Tregs that express CDH1-CAR or overexpress CD103 prevent and treat GVHD in a humanized mouse model and in patients with acute GVHD, while effector T cells that express CDH1-CAR or overexpress CD103 exacerbate GVHD in humanized mouse models and in patients with acute GVHD.

FIG. 2 contains a schematic of an exemplary method for expanding and/or engineering Tregs. T cells are isolated from healthy donors, CD4+CD25+FOXP3+ cells are isolated using negative selection beads (STEM CELL). T cells were then stimulated with CD3/CD28 beads on day 0, cultured in the presence of IL-2, and transduced with lentivirus express the transgene of interest on day 1 (CDH1-CAR or CD103). T cell expansion can last for a period of 6-14 days or 7-21 days. At the end of expansion, flow cytometric analysis confirmed that Tregs maintained their phenotype and suppressive functions. Cryopreserved Tregs were used for in vitro and in vivo experiments. Prior to their use, cells were thawed and rested for 12 hours.

FIGS. 3A and 3B contain graphs showing that Tregs isolated from human peripheral blood mononuclear cells (PBMCs) were expanded in vitro (FIG. 3A) with high purity (FIG. 3B).

FIG. 4 contains a graph showing that Jurkat cells transduced with lentivirus particles encoding a CDH1-CAR nucleic acid expressed a CAR containing hSC10.17 clone (CDH1-specific scFv) linked to a CD8 hinge (CD8 H), a CD28 transmembrane domain (CD28 TM), and a CD3zeta signaling domain (CD3z).

FIG. 5 contains a schematic of an exemplary humanized mouse model for evaluating CDH1-CAR therapy.

FIG. 6. Regulatory T cells suppress effector T cell proliferation. Effector T cells were labeled with CFSE and mixed with regulator t cells isolated from the same donor, in the presence of stimulatory beads (4:1 or 1:1 ratio). The 4:1 ratio is to expand effector T cells, and 1:1 ratio is to activate T cells. Under both conditions, regulatory T cells inhibit the expansion of effector T cells, which is dependent on the cell dose. The higher the ratio of regulatory T cells to effector T cells, the more profound is the inhibition of effector T cells.

FIG. 7. Regulatory T cells suppress effector CD8⁺ T cell proliferation. Effector T cells were labeled with CFSE and mixed with regulatory T cells isolated from the same donor, in the presence of stimulatory beads (4:1 or 1:1 ratio). The 4:1 ratio is to expand effector T cells, and 1:1 ratio is to activate T cells. Under both conditions, regulatory T cells inhibit the expansion of effector T cells, which is dependent on the cell dose. The higher the ratio of regulator T cells to effector T cells, the more profound is the inhibition of effector CD8⁺ T cells.

FIG. 8 shows a schematic of an exemplary method for treatment and/or prevention of GVHD through modulation of the interaction between CDH1 on epithelial cells and its ligand (CD103) on T-cells. This method involves a xenograft model of a luciferase positive human epithelial cell line (MCF7). Mice are injected with CDH1-CAR-Teff, which has a killing effect on the xenograft, and are randomized to receive one of the following treatments to suppress the killing effect: 1) CDH1-CAR-Treg, 2) CD103^{high}-Treg, 3) UTD-Treg, and 4) saline. The killing effect on the xenograft is measured and compared between groups using serial bioluminescence imaging on days 5, 10, 15, and 20.

FIG. 9 shows a schematic of another exemplary method for treatment and/or prevention of GVHD through modulation of the interaction between CDH1 on epithelial cells and its ligand (CD103) on T-cells. This method involves a xenograft model of human hematopoietic cells, where non-irradiated NSG (NOD-SCID-γ chain^{-/-}) mice are injected with 10 million human PBMC. These mice will normally develop xenogeneic GVHD after approximately 4-5 weeks. To prevent GVHD, the mice are also randomized to receive one of the following treatments: 1) anti-CD103-CAR-Teff, 2) CD103^{high}_Treg, 3) UTD-Treg, and 4) saline. The GVHD in the mice is assessed through serial measurement of the mice every 2-3 days.

FIG. 10A-10D are flow cytometry plots showing isolation of Tregs (FIGS. 10C and 10D) from PBMCs (FIGS. 10A and 10B) as indicated by various markers. Tregs make up about 5% of peripheral T-cells. Typical phenotypic markers of Tregs are CD4+, CD25+, and FoxP3+, but since FoxP3 is an intracellular marker, CD127 low expression is used instead.

FIGS. 11A and 11B are flow cytometry plots showing isolation of natural Tregs. The stability of Tregs after in vitro expansion is a known issue (Hoffmann et al., Blood, 108 (13): 4260-4267 (2006)). To overcome this issue, the specific subpopulation of natural Tregs that are characterized by CD45RA+/CD4+/CD25+/CD127 low expression was isolated.

FIG. 12 is a graph plotting in vitro Treg expansion, which was achieved by stimulating Tregs with anti-CD3/CD28 beads at a 3:1 bead to cell ratio supplemented with IL2 (400 IU/ml) for one week, followed by resting condition (no beads and low dose IL2 at 100 IU/ml), followed by a second stimulation with anti-CD3/CD28 beads at a 1:1 bead to cell ratio supplemented with IL2 (400 IU/ml) for another week. Using this technique, a 30- to 300-fold expansion was achieved at the end of 21 days.

FIG. 13A is a schematic showing the structure of a second generation anti-CDH1 scFv CAR construct with a CD28 co-stimulatory signal. FIGS. 13B-13E are flow cytometry plots showing production of CDH1-CAR-T cells. Teffs (FIGS. 13B and 13C) and Tregs (FIGS. 13D and 13E) were transduced with the construct shown in FIG. 13A, using lentivirus.

FIGS. 14A-14D are flow cytometry plots showing CD103^{high} T-cell production. Lentivirus technology similar to a first generation CAR was used to generate Teffs (FIGS. 14A and 14B) and Tregs (FIGS. 14C and 14D) with high expression of CD103, without the co-stimulatory signals.

FIGS. 15A and 15B are flow cytometry plots showing Treg purity at day 0 (FIG. 15A) and after in vitro expansion for 9 days (FIG. 15B). These studies demonstrated that Tregs maintained their regulatory phenotype at the end of expansion.

FIGS. 16A and 16B are graphs plotting Treg suppressive function in vitro. Effector cells were stained with CFSE, stimulated with anti-CD3/CD28 beads, and co-cultured with varying ratios of Tregs, and the number of proliferating cells was measured after four days. This was tested on day 0 (FIG. 16A) with freshly isolated Tregs and at day 9 (FIG. 16B) at the end of the first expansion. As shown in both graphs, proliferation of effector cells was decreased with higher ratios of Tregs. Notably, the suppressive function was more pronounced with the use of in vitro expanded Tregs.

FIG. 17 is a graph plotting CDH-1-CAR-Treg function in vitro. To demonstrate CDH-1 specific suppressive function of Tregs, a proliferation assay was conducted in which CAR19 (used as effector cells) was co-cultured with different ratios of CDH1-CAR-Treg. This allowed the use of a different stimulus for CAR19 than for Tregs. As shown in the graph, CDH1-CAR-Tregs exerted their suppressive function on CAR19 in the presence of MCF7 cells but not in the absence of MCF7 cells.

FIG. 18 is a graph plotting CD103^{high} in vitro function. The CD103^{high} Tregs had more potent suppressive function than transduced Tregs when they were co-cultured with effector T cells.

FIG. 19 is an image showing CDH1-CAR-teff and CD103^{high}-Teff function in vitro. In particular, the in vitro function of CDH-1-CAR-Teffs and CD103^{high} Teffs was assessed in a killing assay, in which luciferase+human epithelial cells (in this case MCF7) were mixed with different ratios of CDH1-CAR-Teffs or CD103^{high} Teffs. After 48 hours, a significant killing effect was observed from CDH1-CAR-Teff, but not from UTD or CD103^{high} Teffs.

FIG. 20A shows bioluminescent imaging of NSG mice, demonstrating successful engraftment of MCF7 tumor cells one week after subcutaneous injection (left image) and eradication of MCF7 tumors 7 days after treatment with CDH1-CAR-Teffs (right image).

FIG. 20B is a graph plotting of tumor burden of MCF7 xenografts treated with untransduced T cells, CDH-1-CAR Teff, or CD103^{high} Teff cells, measured by bioluminescence imaging.

FIG. 21 is a graph plotting data from a xenograft model of GVHD, in which non-irradiated NSG mice were injected with 10 million PBMC and monitored for development of GVHD. Mice began to develop signs of acute GVHD around day 28, as demonstrated by their rapid weight loss after day 28 in the graph.

FIG. 22 depicts the general structure of an anti-CD103 CAR construct in which an anti-CD103 scFv was built into a second generation CAR with a CD28 co-stimulatory signal. The sequences encoding the light and heavy chains (SEQ ID NOS: 9 and 10, respectively) are provided below the structure.

FIG. 23 is a flow cytometry plot showing anti-CD103-CAR transduction; effector T-cells were successfully transduced with anti-CD103-CAR using lentivirus technology.

### DETAILED DESCRIPTION

This document provides methods and materials involved in treating a mammal (e.g., a human) having or at risk of developing GVHD. For example, one or more T cells (e.g., one or more Tregs) expressing one or more antigen receptors targeting one or more epithelial-specific antigens can be administered (e.g., by adoptive transfer) to a mammal having GVHD (or at risk of developing GVHD) to treat the GVHD (or to reduce the risk of developing GVHD). In some cases, a CAR targeting an epithelial-specific antigen (e.g., CDH1) can be expressed on a Treg to target the Treg to epithelial tissues. For example, one or more T cells expressing one or more CARs targeting CDH1 (e.g., CDH1-CARs) can be administered (e.g., by adoptive transfer) to a mammal having GVHD (or at risk of developing GVHD) to treat the GVHD (or to reduce the risk of developing GVHD). In some cases, a CD103 polypeptide targeting an epithelial-specific antigen (e.g., CDH1) can be expressed on a Treg to target the Treg to epithelial tissues. For example, one or more T cells expressing one or more CD103 polypeptides can be administered (e.g., by adoptive transfer) to a mammal having GVHD (or at risk of developing GVHD) to treat the GVHD (or to reduce the risk of developing GVHD). In some cases, a CD103 polypeptide targeting an epithelial-specific antigen (e.g., CDH1) can be expressed on a Treg to target the Treg to epithelial tissues to treat an epithelial disease and/or an autoimmune disease (e.g., an autoimmune disease that involves epithelial inflammation in, for example, skin, gut tissue, and/or liver tissue.

A T cell described herein (e.g., a T cell expressing epithelial-specific antigen receptors) can be any appropriate T cell. In some cases, a T cell can be a naïve T cell. In some cases, a T cell can be an immunosuppressive T cell. An example of T cells that can be used as described herein includes, without limitation, Tregs. For example, a T cell expressing one or more antigen receptors targeting one or more epithelial-specific antigens can be a Treg. In some cases, the methods and materials described herein can be used to redirect CAR T cells with an inhibitory signal or to redirect stem cells (e.g., mesenchymal stem cells).

A T cell described herein can express (e.g., can be engineered to express) any appropriate antigen receptor that binds an epithelial-specific antigen (e.g., an antigen present on epithelial cells with minimal, or no, expression on other cell types). For example, a T cell can be engineered to express an antigen receptor that targets an antigen (e.g., a cell surface antigen) expressed by epithelial cells in a mammal having or at risk of developing GVHD. In some cases, an antigen receptor can be a heterologous antigen receptor. In some cases, an antigen receptor can be a CAR. In some cases, an antigen receptor can be a recombinant antigen receptor. In some cases, an antigen receptor can include an antibody or a fragment thereof. For example, an antigen receptor can include an antigen-binding (Fab) fragment from an antibody that targets an epithelial-specific antigen. In some cases, an antigen receptor can include one or more variable regions of the heavy (VH) chains and one or more variable regions of the light (VL) chains (e.g., as a recombinant protein or a fusion protein) from an antibody that targets an epithelial-specific antigen. For example, an antigen receptor can be a single chain variable fragment (scFv) that targets an epithelial-specific antigen. Examples of antigen receptors that bind an epithelial antigen include, without limitation, CD103, hSC10.17, CD234, EPCAM, EMA, MUC1, cytokeratin, CA125, ALCAM, HLA, Desmin, Eputheliam Antigen antibody, CD227, ESA, Galactin 3, GGT, HLA-DR, Lectin, LAMP-1, MMR, MOC-31, p16, p63, p-Cadherin, PSA, surfactant, Transthyretin, VAT-1, and Vimentin. For example, a T cell engineered to target epithelial tissues can express a CD103 polypeptide. In such cases, the engineered T cell can include exogenous nucleic acid that encodes and expresses the CD103 polypeptide. As another example, a T cell engineered to target epithelial tissues can express an antibody or antibody fragment of an anti-CDH1 antibody (e.g., an hSC10.17 scFv). In such cases, the engineered T cell can include exogenous nucleic acid that encodes and expresses the hSC10.17 scFv. In some cases, a T cell engineered to target epithelial tissues can express both a CD103 polypeptide and an antibody or antibody fragment of an anti-CDH1 antibody (e.g., an hSC10.17 scFv).

An epithelial antigen can be any appropriate epithelial antigen. An epithelial antigen can be expressed on any appropriate type of epithelial cell (e.g., skin cells, gastrointestinal tract cells, lung cells, liver cells, brain cells, kidney cells, ovarian cells, uterus cells, bladder cells, and pancreatic cells). In some cases, an epithelial antigen can be a cell adhesion molecule (CAM). Examples of epithelial antigens include, without limitation, CDH1, CD103, hSC10.17, CD234, EPCAM, EMA, MUC1, cytokeratin, CA125, ALCAM, HLA, Desmin, Eputheliam Antigen antibody, CD227, ESA, Galactin 3, GGT, HLA-DR, Lectin, LAMP-1, MMR, MOC-31, p16, p63, p-Cadherin, PSA, surfactant, Transthyretin, VAT-1, and Vimentin. For example, a T cell engineered to target epithelial tissues can bind to CDH1. In some cases, a T cell can be engineered to express a CDH1-CAR to target CDH1 expressed by epithelial cells in a mammal having or at risk of developing GVHD. In some cases, a T cell can be engineered to express a CD103 polypeptide to target CDH1 expressed by epithelial cells in a mammal having or at risk of developing GVHD.

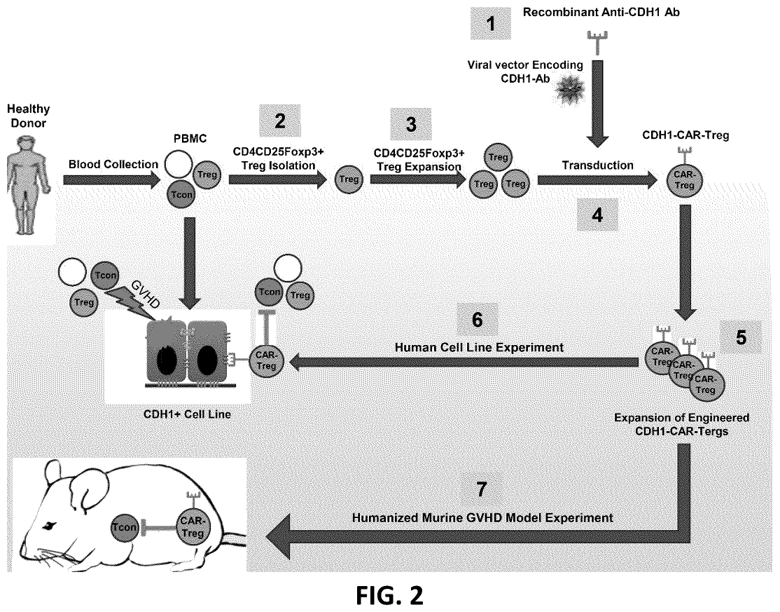

Any appropriate method can be used to express an antigen receptor described herein (e.g., an antigen receptor targeting one or more epithelial-specific antigens) on a T cell described herein. For example, a nucleic acid encoding an antigen receptor can be introduced into a T cell. In some cases, a nucleic acid encoding an antigen receptor can be introduced into a T cell by transduction (e.g., viral transduction) or transfection. In some cases, a nucleic acid encoding an antigen receptor can be introduced ex vivo into one or more T cells. For example, ex vivo engineering of T cells to express an antigen receptor can include transducing isolated T cells with a lentiviral vector encoding an antigen receptor. In cases where T cells are engineered ex vivo to express an antigen receptor, the T cells can be obtained from any appropriate source (e.g., a mammal such as the mammal to be treated or a donor mammal, or a cell line). In some cases, CAR T cells can be prepared as described herein (see, e.g., FIG. 2 and Example 1). For example, a CDH1-CAR can be expressed on a Treg to direct the Treg to epithelial tissues by introducing one or more constructs containing a nucleic acid encoding the CAR (e.g., a CAR targeting CDH1) into the Treg. In some cases, CAR T cells can be prepared as described elsewhere (see, e.g., Blat et al., Mol. Ther., 22 (5): 1018-28 (2014); MacDonald et al., J. Clin. Invest., 126 (4): 1413-24 (2016); and Yoon et al., Blood, 129 (2): 238-245 (2017)).

Also provided herein are CARs and constructs (e.g., nucleic acid constructs) encoding CARs described herein (e.g., CARs targeting an epithelial-specific antigen (e.g., CDH1)). For example, a construct encoding a CAR targeting CDH1 (e.g., a CDH1-CAR) can include a nucleic acid sequence encoding one or more molecules that bind CDH1 described herein. In some cases, a CDH1-CAR can include an anti-CDH1 antibody (e.g., hSC10.17) heavy chain and an anti-CDH1 antibody (e.g., hSC10.17) light chain. A CAR described herein (e.g., a CDH1-CAR) also can include one or more additional components. Examples of additional components that can be included in a CAR include, without limitation, a leader sequence (e.g., a CD8 leader sequence), a hinge (e.g., a CD8 hinge), a transmembrane domain (e.g., a CD8 transmembrane domain or a CD28 transmembrane domain), a signaling domain such as a CD3zeta signaling domain or a signaling domain used to generate a third generation CART or CAR Treg that increases immunosuppression that may increase secretion of immunosuppressive cytokines, such as PD1, CTLA4, TIM3, or other inhibitory molecules. In some cases, nucleic acid encoding a component of a construct encoding a CAR can be separated from nucleic acid encoding another component using one or more linkers. Nucleic acids in a construct encoding a CAR can be present in any order. For example, constructs encoding a CHD1-CAR can be generated in a light to heavy orientation of the scFv or in a heavy to light orientation of the scFv. Exemplary nucleic acid sequences for some additional components that can be included in a construct described herein are as follows.

CD8 leader sequence
(SEQ ID NO: 1)
ATGGCCTTACCAGTGACCGCCTTGCTCCTGCCGCTGGCCTTGCTGCTCCA
CGCCGCCAGGCCG
hSC10.17 heavy chain (HC)
(SEQ ID NO: 2)
GAGGTGCAGCTGGTGGAGTCTGGGGGAGGCTTGGTACAGCCTGGGGGGTC
CCTGAGACTCTCCTGTGCAGCCTCTGGATTCACCTTCAGTAGCTATGGCA
TGCACTGGGTCCGCCAGGCTCCAGGGAAGGGGCTGGAGTGGGTTGCATAC
ATTACTACTAGAAGTAGTACCATATACTACGCAGACTCTGTGAAGGGCCG
ATTCACCATCTCCAGAGACAATGCCAAGAACTCACTGTATCTGCAAATGA
ACAGCCTGAGAGCCGAGGACACGGCTGTGTATTACTGTACTAGAGAACCC
CTAACTGGATACTATGCTATGGACTACTGGGGTCAAGGAACCTCAGTCAC
CGTCTCCTCAG
Linker
(SEQ ID NO: 3)
GGAGGTGGCGGATCAGGCGGAGGAGGCAGCGGCGGAGGTGGATCAGGAGG
CGGAGGGTCA
hSC10.17 light chain (LC)
(SEQ ID NO: 4)
GATGTTGTGATGACTCAGTCTCCACTCTCCCTGCCCGTCACCCTTGGACA
GCCGGCCTCCATCTCCTGCAGGTCTAGTCAAAGCATCGTACACAGTGATG
GAAACACCTACTTGGAATGGTATCAGCAGAGGCCAGGCCAATCTCCAAGG
CGCCTAATTTATAAGGTTTCTAACCGGTTCTCTGGGGTCCCAGACAGATT
CAGCGGCAGTGGGTCAGGCACTGATTTCACACTGAAAATCAGCAGGGTGG
AGGCTGAGGATGTTGGGGTTATTACTGCTTTCAAGGTTCACATGCTCCGT
GGACGTTCGGTGGAGGCACCAAGGTGGAAATCAAAC
CD8 hinge
(SEQ ID NO: 5)
ACCACGACGCCAGCGCCGCGACCACCAACACCGGCGCCCACCATCGCGTC
GCAGCCCCTGTCCCTGCGCCCAGAGGCGTGCCGGCCAGCGGCGGGGGGCG
CAGTGCACACGAGGGGGCTGGACTTCGCCTGTGAT
CD8 transmembrane domain
(SEQ ID NO: 6)
ATCTACATCTGGGCGCCCTTGGCCGGGACTTGTGGGGTCCTTCTCCTGTC
ACTGGTTATCACCCTTTACTGC
CD28 signaling domain
(SEQ ID NO: 7)
AGGAGTAAGAGGAGCAGGCTCCTGCACAGTGACTACATGAACATGACTCC
CCGCCGCCCCGGGCCCACCCGCAAGCATTACCAGCCCTATGCCCCACCAC
GCGACTTCGCAGCCTATCGCTCC
CD3zeta signaling domain
(SEQ ID NO: 8)
AGAGTGAAGTTCAGCAGGAGCGCAGACGCCCCCGCGTACAAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCA
AGGGGCACGATGGCCTTTACCAGGGTCTCAGTACAGCCACCAAGGACACC
TACGACGCCCTTCACATGCAGGCCCTGCCCCCTCGC

In some cases, a nucleic acid construct encoding a CDH1-CAR can include a CD8 leader sequence, a hSC10.17 heavy chain, a linker, a hSC10.17 light chain, a CD8 hinge, a CD28 transmembrane domain, and a CD3zeta signaling domain. For example, a nucleic acid construct encoding a CHD1-CAR can include a CD8 leader including the sequence set forth in SEQ ID NO:1, a hSC10.17 heavy chain including the sequence set forth in SEQ ID NO:2, a linker including the sequence set forth in SEQ ID NO:3, a hSC10.17 light chain including the sequence set forth in SEQ ID NO:4, a CD8 hinge including the sequence set forth in SEQ ID NO: 5, a CD28 transmembrane domain including the sequence set forth in SEQ ID NO:7, and a CD3zeta signaling domain including the sequence set forth in SEQ ID NO:8.

This document also provides materials and methods for treating mammals (e.g., humans) having (or at risk of developing) GVHD. For example, T cells described herein (e.g., T cells expressing epithelial-specific antigen receptors) can be administered (e.g., by adoptive transfer) to a mammal having or at risk of developing GVHD to reduce the severity of GVHD within the mammal. In some cases, reducing the severity of GVHD in a mammal can include reducing or eliminating one or more symptoms of GVHD (e.g., skin rashes, immune-mediated pneumonitis, intestinal inflammation, sloughing of the intestinal mucosal membrane, severe diarrhea, abdominal pain, nausea, vomiting, and/or elevated bilirubin levels). In some cases, reducing the severity of GVHD in a mammal can include reducing the stage of GVHD. The stage of GVHD can be evaluated as described elsewhere (see, e.g., Jacobsohn et al., Orphanet. J. Rare Dis., 2:35 (2007)).

Any appropriate mammal having (or at risk of developing) GVHD can be treated as described herein. For example, T cells described herein (e.g., T cells expressing epithelial-specific antigen receptors) can be administered (e.g., by adoptive transfer) to humans and other primates such as monkeys having (or at risk of developing) GVHD to treat the GVHD. In some cases, dogs, cats, horses, cows, pigs, sheep, mice, and rats can be treated as described herein.

Any appropriate GVHD can be treated as described herein. GVHD can be acute GVHD (aGVHD) or chronic GVHD. GVHD can be allogeneic GVHD (allo-GVHD) or autologous GVHD (auto-GVHD). GVHD can be any stage of GVHD. In some cases, GVHD can be associated with (e.g., following) a transplant. A transplant can be an allogeneic transplant such as HCT. A transplant can be autologous such as autologous hematopoietic progenitor cell transplantation (HPCT).

In some cases, a mammal can be identified as having (or as being at risk of developing) GVHD. Any appropriate method for identifying a mammal as having (or as being at risk of developing) GVHD can be used. Once identified as having (or as being at risk of developing) GVHD, the mammal can be administered (e.g., by adoptive transfer) or instructed to self-administer one or more T cells described herein (e.g., T cells expressing epithelial-specific antigen receptors) to treat the GVHD within the mammal.

Any appropriate method can be used to administer T cells described herein (e.g., T cells expressing epithelial-specific antigen receptors) to a mammal (e.g., a mammal having, or at risk of developing, GVHD). Examples of methods of administering T cells described herein to a mammal can include, without limitation, injection (e.g., IV, ID, IM, or subcutaneous injection). For example, T cells expressing epithelial-specific antigen receptors can be administered to a human by intravenous injection.

In some cases, a mammal having (or at risk of developing) GVHD being treated as described herein (e.g., by administration of one or more T cells described herein (e.g., T cells expressing an epithelial-specific antigen receptors)) also can be treated with one or more therapeutic agents. A therapeutic agent used in combination with T cells described herein can be any appropriate therapeutic agent. In some cases, a therapeutic agent can be a GVHD agent. In some cases, a therapeutic can be an immunosuppressive agent. Examples of therapeutic agents that can be used in combination with T cells described herein include, without limitation, systemic steroids (e.g., corticosteroids), topical steroids, infliximab, tocilizumab, natalizumab, ibrutinib, ruxolitinib, immunoglobulins (e.g., anti-thymocyte globulin (ATG)), ECP (extracoporeal photopheresis), TNF-α blocking agents, alemtuzumab, IVIG, calcineurin inhibitors (e.g., tacrolimus and/or cyclosporine), sirolimus, IL-2 blocking agents, low dose IL-2, mycophenolae mofetil, pentostatin, T cell depleting chemotherapy, rituximab, brentuximab, and mesenchymal stem cells.

This document also provides kits containing one or more materials described herein. For example, materials provided in kits described herein can be used for treating mammals (e.g., humans) having (or at risk of developing) GVHD as described herein.

In some cases, one or more T cells described herein (e.g., T cells expressing an epithelial-specific antigen receptors) can be combined with packaging material and sold as a kit. The packaging material included in such a kit typically contains instructions or a label describing how the composition can be used, for example, in an adoptive transfer to treat GVHD as described herein. In some cases, one or more constructs (e.g., nucleic acid constructs) described herein (e.g., encoding CARs that bind one or more epithelial-specific antigens) can be combined with packaging material and sold as a kit. The packaging material included in such a kit typically contains instructions or a label describing how the composition can be used, for example, to express one or more CARs in T cells (e.g., Tregs) to engineer the T cells to express CARs that bind one or more epithelial-specific antigens (e.g., CDHa-CARs). A kit also can include instructions or a label describing how the engineered T cells can be used, for example, in adoptive transfer to treat GVHD as described herein. A kit also can include materials for use in an adoptive transfer procedure.

The invention will be further described in the following examples, which do not limit the scope of the invention described in the claims.

### EXAMPLES

### Example 1: Targeting E-Cadherin (CDH1) with Chimeric Antigen Receptor Regulatory T-cells for the Treatment of Acute Graft-Versus-Host Disease

### Methods

### Cell Lines and Primary Samples

Cell lines were obtained from ATCC (K-562, MCF-7, and NALM6). All cell lines were tested for sterility before experiments. For some experiments, MCF-7 and NALM-6 cells were transduced with zsGreen/GFP lentivirus and then sorted to obtain >99% positive population. Cell lines MOLM-14 and K562 were used as controls as indicated in the relevant figures. The cell lines were maintained in culture with RPMI1640 (Gibco, 11875-085, LifeTechnologies) supplemented with 10% FBS (Gemini, 100-106) and 50 U/mL penicillin/streptomycin (Gibco, Life Technologies, 15070-063). For all functional studies, primary cells were thawed at least 12 hours before experiment and rested at 37° C.

### Generation of CAR Constructs and CAR T Cells

The anti-CDH1 chimeric antigen receptor (single chain variable fragment derived from clone hSC10.17, CD8 hinge, 4-1BB costimulatory domain and CD3 zeta signaling domain) was generated de novo and cloned into a third generation lentivirus. Normal donor T regulatory cells were selected using negative selection Kit (Stem Cell), and expanded in vitro using anti-CD3/CD28 Dynabeads (Invitrogen, Life Technologies, Grand Island, NY, USA, added on the first day of culture) and IL-2 at different concentrations (100, 500 and 1000 IU/mL). T cells were transduced with lentiviral supernatant one day following stimulation at a multiplicity of infection of 3. The anti-CD3/CD28 Dynabeads were removed on day 6, and T cells were grown in T-cell media (X-vivo 15 media, human serum 5%, penicillin, streptomycin and glutamax). CART cells were then cryopreserved on day 8 for future experiments. Prior to all experiments, T cells were thawed and rested overnight at 37° C.

### Multiparametric Flow Cytometry

Anti-human antibodies were purchased from Biolegend (San Diego, CA, USA), eBioscience (San Diego, CA, USA), or BD Biosciences (San Jose, CA, USA). Cells were isolated from in vitro culture or from animals, washed once in phosphate-buffered saline supplemented with 2% fetal calf serum, and stained at 4° C. after blockade of Fc receptors. For cell number quantitation, Countbright beads (Invitrogen) were used according to the manufacturer's instructions (Invitrogen). In all analyses, the population of interest was gated based on time gating, followed by forward vs side scatter characteristics, followed by singlet gating, and live cells were gated using Live Dead Aqua (Invitrogen). Surface expression of anti-CDH1 CAR was detected by staining with an Alexa Fluor 647-conjugated goat anti-mouse F(ab′)2 antibody from Jackson Immunoresearch (West Grove, PA, USA) or with protein L. Flow cytometry was performed on a four-laser analyzer (BD Canto-II). All analyses were performed using FlowJo X10.0.7r2.

### T-Cell Function Assays

### T-Cell Degranulation and Intracellular Cytokine Assays

Briefly, T cells were incubated with target cells at a 1:5 ratio. After staining for CAR expression, antibodies against CD107a, CD28, CD49d and monensin were added at the time of incubation. After 4 hours, cells were harvested and stained for CD3 and Live Dead staining (Invitrogen). Cells were fixed and permeabilized (FIX & PERM Cell Fixation & Cell Permeabilization Kit, Life Technologies), and intracellular cytokine staining was then performed as indicated in the specific experiments.

### Proliferation Assays

T cells were washed and re-suspended at 1×10⁷/mL in 100 μL of phosphate-buffered saline and labeled with 100 μL of carboxyfluorescein succinimidyl ester (CFSE) 2.5 μM (Life Technologies) for 5 minutes at 37° C. The reaction was then quenched with cold R10, and the cells were washed three times. Targets were irradiated at a dose of 100 Gy. T cells were incubated at a 1:1 ratio with irradiated target cells for 120 hours. Cells were then harvested, stained for CD3, CAR and Live Dead aqua (Invitrogen), and Countbright beads (Invitrogen) were added prior to flow cytometric analysis.

### Treg Suppressive Assays

Regulatory T cells were co-cultured with CFSE labeled effector T cells, in the presence of low concentrations of CD3/CD28 beads, for 5 days. Proliferation of CFSE labeled T effector cells was calculated by flow cytometry using CountBright beads for absolute counting.

The suppressive effect of regulatory T cells on CART cell functions was measured through the incubation of regulatory T cells with CFSE labeled CART19 cells, in the presence of their targets. Proliferation of CART cells was analyzed by flow cytometry using CountBright beads for absolute counts.

### Cytotoxicity Assays

MCF-7 cells were used for cytotoxicity assay. In brief, targets were incubated at the indicated ratios with CDH1-CAR T effectors or CDH1-CART regulatory for 4 or 16 hours or 24 to 48 hours. Killing was calculated either by bioluminescence imaging on a Xenogen IVIS-200 Spectrum camera (PerkinElmer, Hopkinton, MA, USA)

### Secreted Cytokine Measurement

Effector and target cells were incubated at a 1:1 ratio in T-cell media for 24 or 72 hours as indicated. Supernatant was harvested and analyzed by 30-plex Luminex array according to the manufacturer's protocol (Millipore).

### In Vivo Experiments

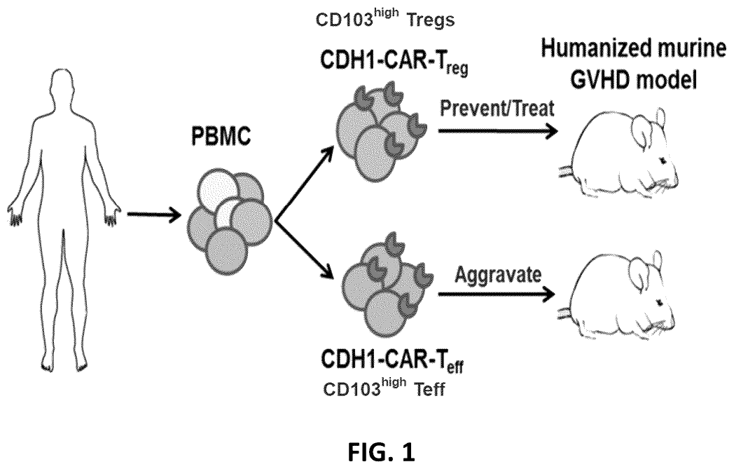

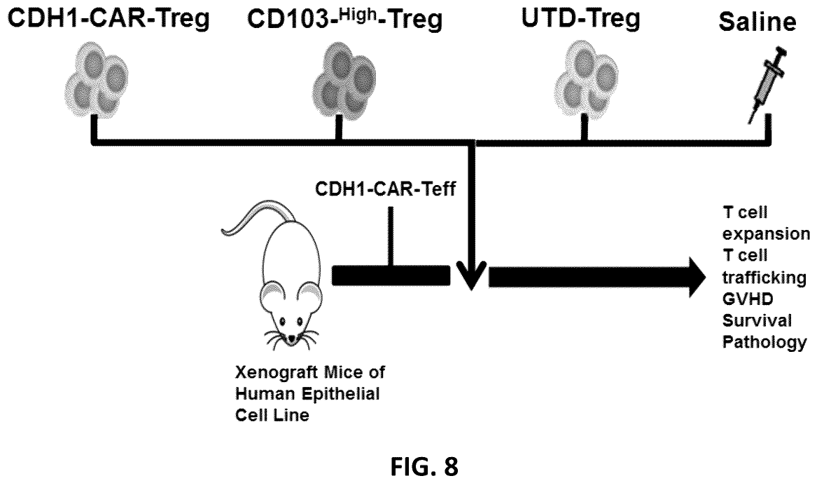

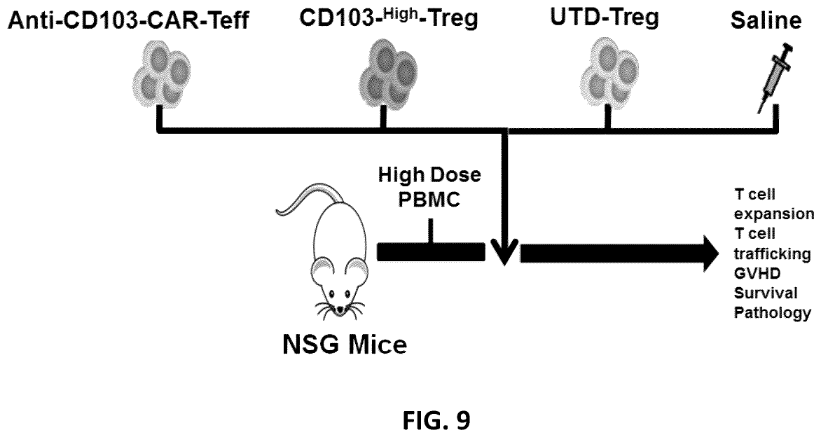

NOD-SCID-γ chain^{-/-} (NSG) originally obtained from Jackson Laboratories were maintained in our laboratory under an IACUC approved breeding protocol. Schemas of the utilized xenograft models are discussed in detail in FIG. 1, FIG. 2, FIG. 8, and FIG. 9. Cells were injected in 200 μL of phosphate-buffered saline at the indicated concentration into the tail veins of mice. Bioluminescent imaging was performed using a Xenogen IVIS-200 Spectrum camera. Images were acquired and analyzed using Living Image version 4.4 (Caliper LifeSciences, Inc., PerkinElmer).

Humanized NSG mice were purchased from the Jackson laboratories. In brief, these mice were injected with CD34⁺ cells as neonates. They develop full human hematopoiesis. After 8 weeks, engraftment was confirmed through bleeding of these mice. They were then treated with CDH1-CAR T effectors or CDH1-CAR T regulatory cells.

### Statistical Analysis

All statistical analyses were performed as indicated using GraphPad Prism 6 for Windows, version 6.04. Student t test was used to compare two groups; in analysis where multiple groups were compared, one-way ANOVA was performed with Holm-Sidak correction for multiple comparisons. When multiple groups at multiple time points/ratios were compared, the Student t test or ANOVA for each time points/ratios was used. Survival curves were compared using the log-rank test. In the figures, asterisks represent P values (*, P<0.05; **, P<0.01; ***, P<0.001; ****, P<0.0001) and “ns” means “not significant” (P>0.05). Further details of the statistics for each experiment are listed in figure legends.

### Results

### Tregs

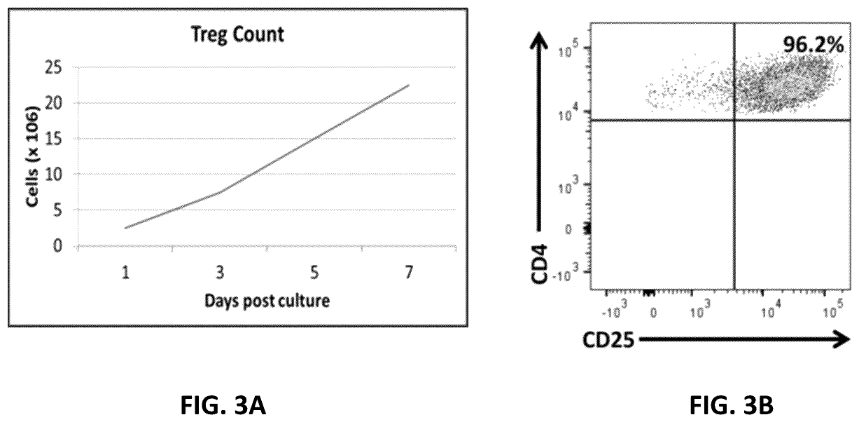

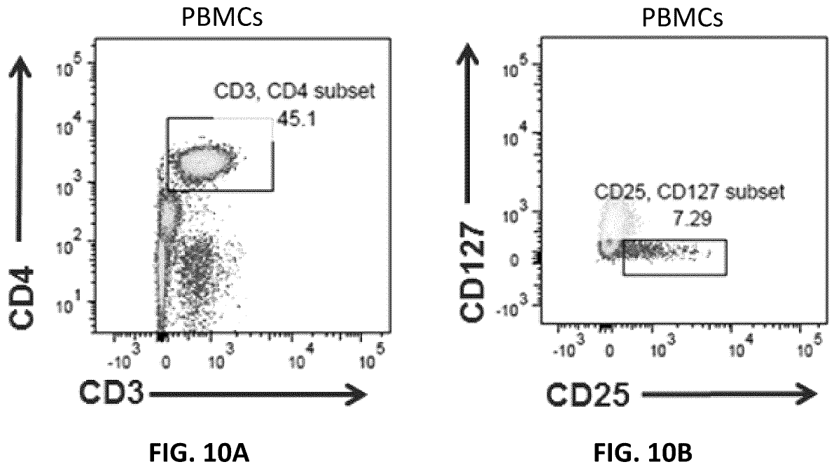

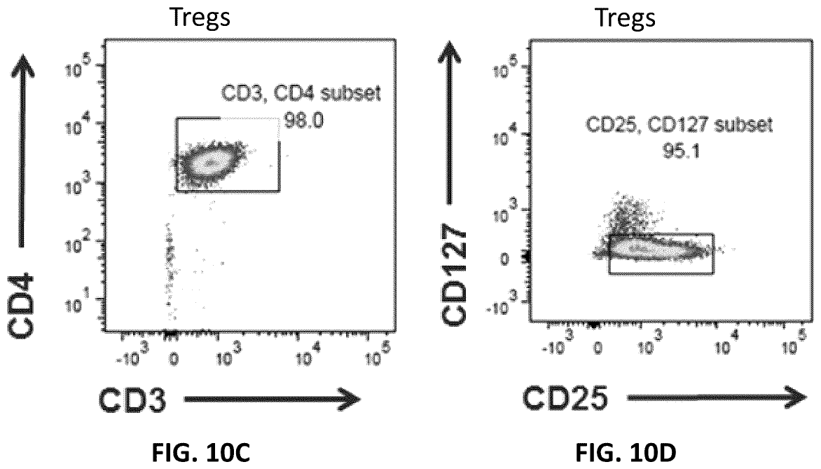

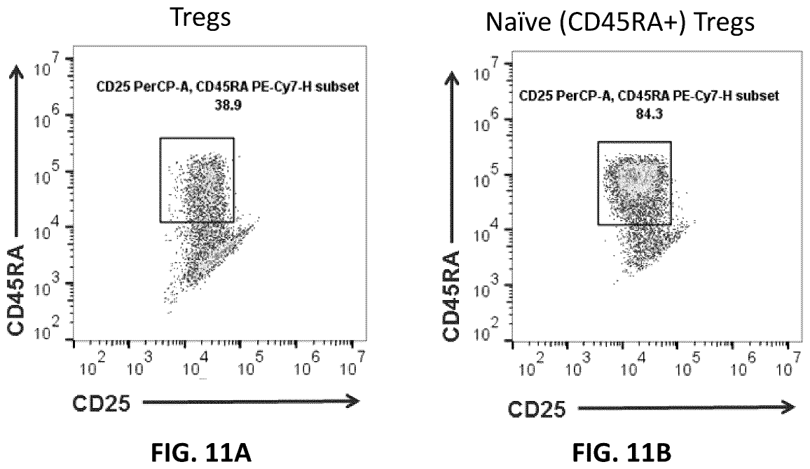

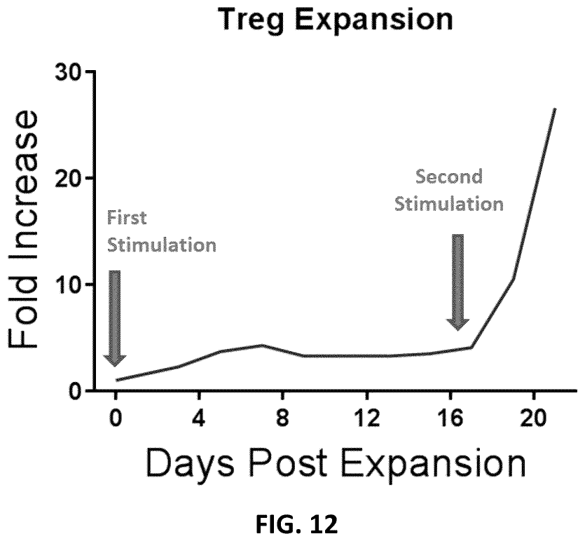

Tregs were isolated from human peripheral blood mononuclear cells (PBMCs), and expanded in vitro with high purity (96%) at day 7. Tregs were expanded using CD3/CD28 and IL-2 for a period of 6-8 days. At the end of expansion, Tregs maintained their phenotype (CD4⁺CD25⁺FOXP3⁺). See, e.g., FIGS. 3A-3B, FIGS. 10A-10D, FIGS. 11A-11B, and FIG. 12.

### CARTregs

A second generation CDH1-CAR was constructed and Jurkat cells were transduced with lentivirus particles encoding the construct.

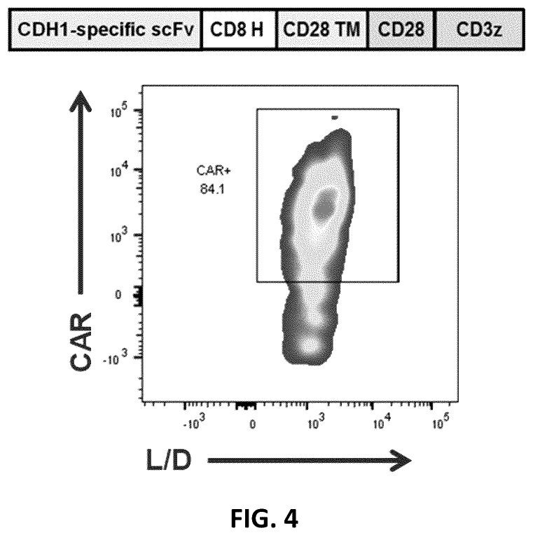

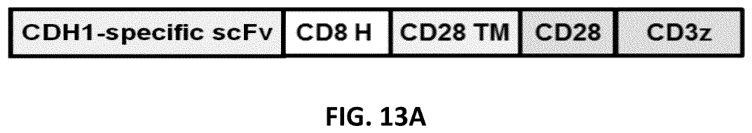

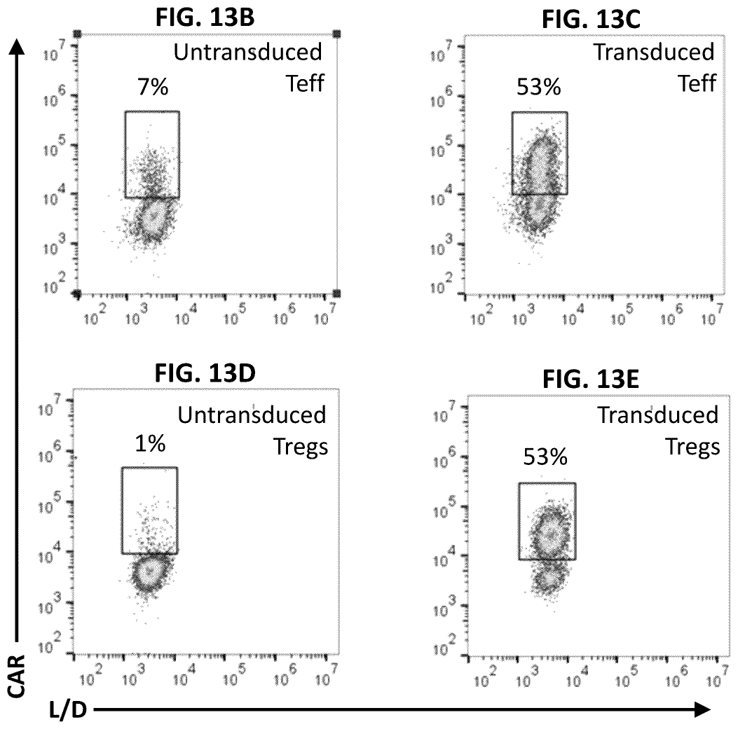

CDH1 CAR was generated de novo and was composed of a hSC10.17 clone linked to a CD8 hinge, a CD28 transmembrane domain, and a CD3zeta signaling domain (see, FIG. 13A). Two constructs were generated using light to heavy and heavy to light orientation of the scFv. T cells were transduced with this construct to generate CDH1-CART cells. See, e.g., FIG. 4.

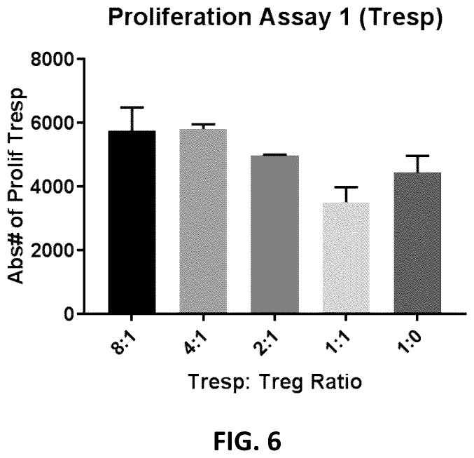

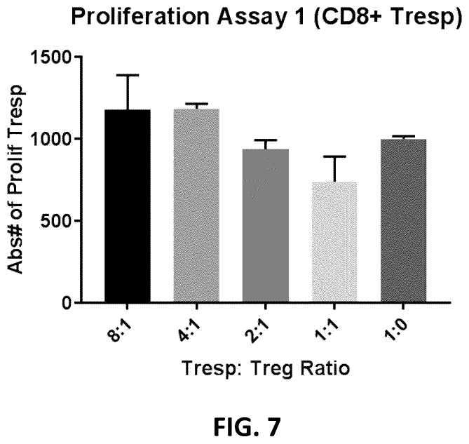

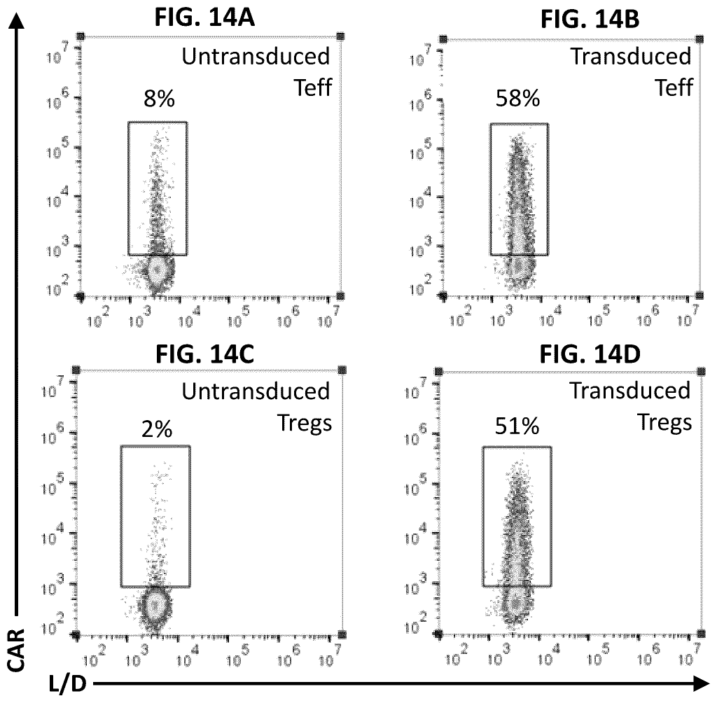

Additional results are presented in FIG. 6, FIG. 7, FIGS. 13A-13E, and FIGS. 14A-14D.

### Example 2: Immunomodulatory Function of CDH1-CAR-Tregs

CDH1-CAR-Treg cells continue to exhibit inhibitory and immunomodulatory functions to treat GVHD.

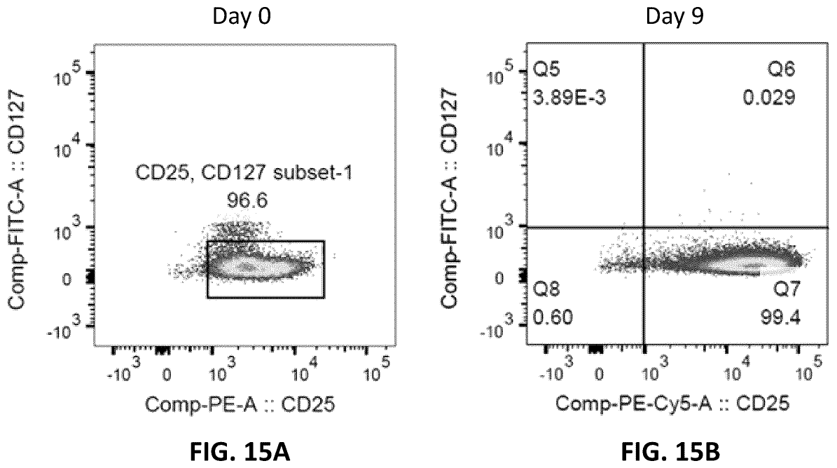

To generate CDH1-CAR-Tregs, ex vivo expanded Tregs are transduced with lentivirus particles encoding CDH1-CAR to generate CDH1-CAR-Tregs, and the purity and immune-phenotype of CDH1-CAR-Tregs before and after expansion were analyzed. CDH1-CAR-T effector (Teff) cells were generated as positive controls. Results are presented in FIGS. 15A-15B.

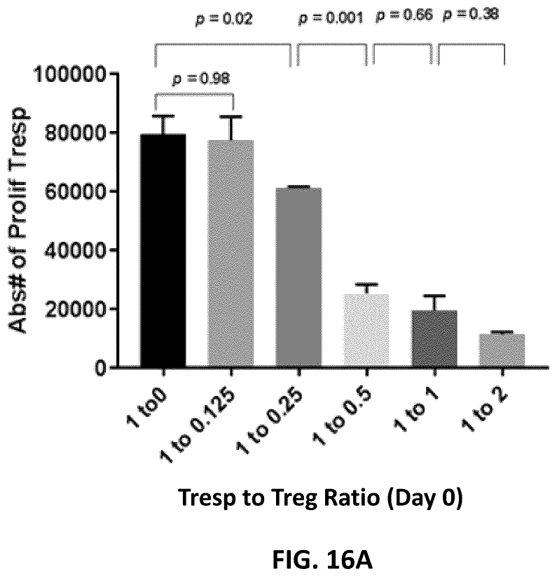

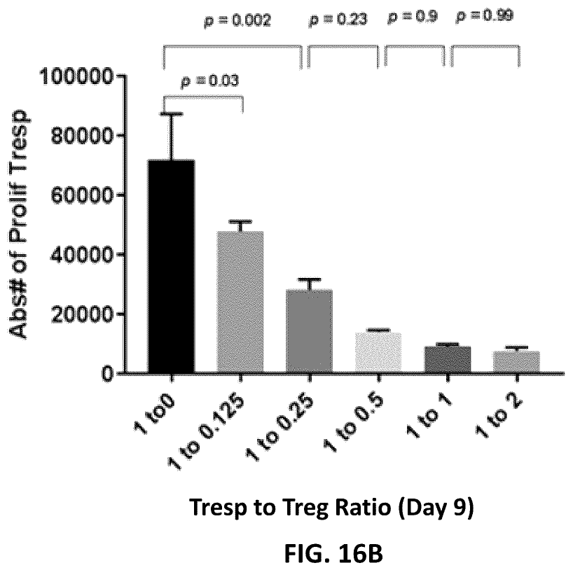

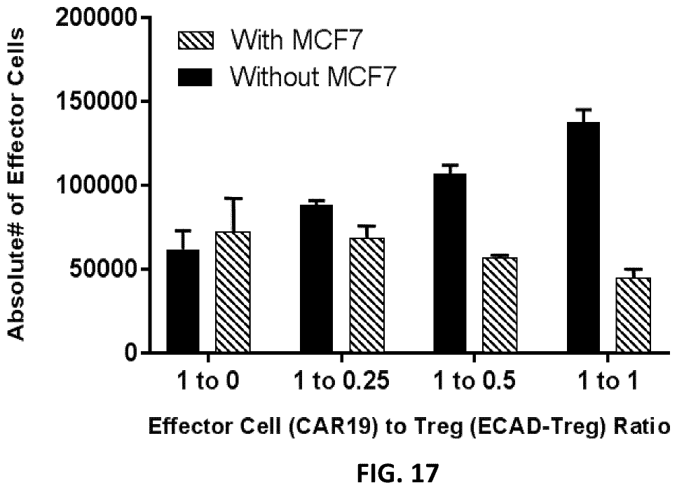

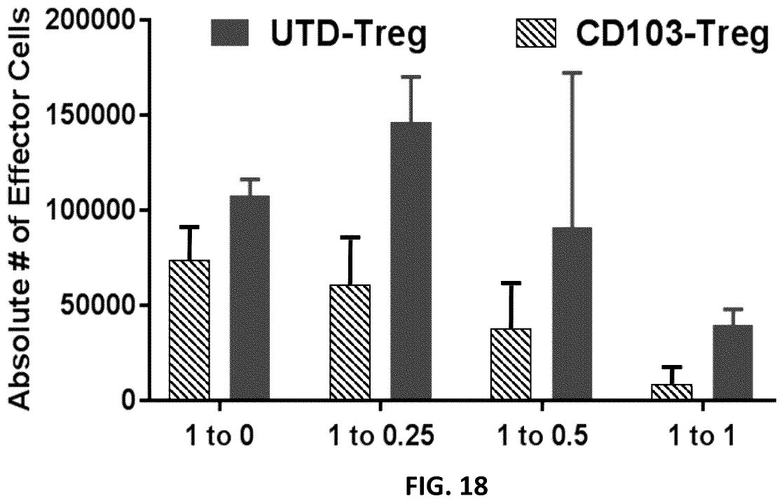

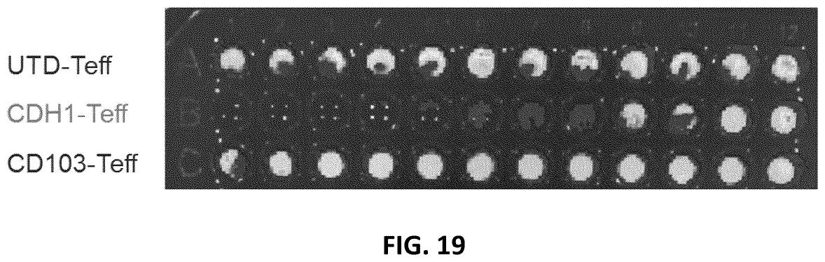

To evaluate the suppressive and immunomodulatory function of ex vivo expanded CDH1-CAR-Treg cells, the proliferation of CDH1-CAR Teff cells in the presence or absence of CDH1-CAR-Treg cells were analyzed. Results are presented in FIGS. 16A-16B, FIG. 17, and FIG. 18. CDH1-CAR Tregs and CDH1-CAR Teff cells were co-cultured with the CDH1⁺ luciferin⁺ cell line MCF7 and cytokine production and the killing of target cells in the co-culture was assessed. Results are presented in FIG. 19. In further studies, cytokine production is measured to evaluate the suppressive and immunomodulatory function of ex vivo expanded CDH1-CAR-Treg cells.

### Example 3: Engineering Tregs to Overexpress CD103

Treg cells overexpressing CD103 polypeptides treat aGVHD.

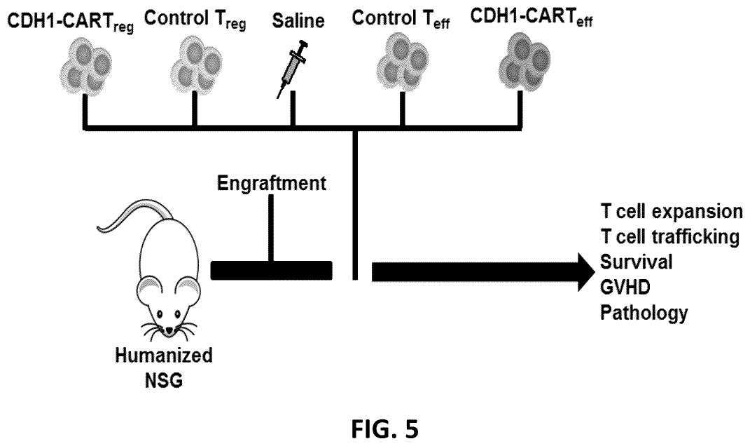

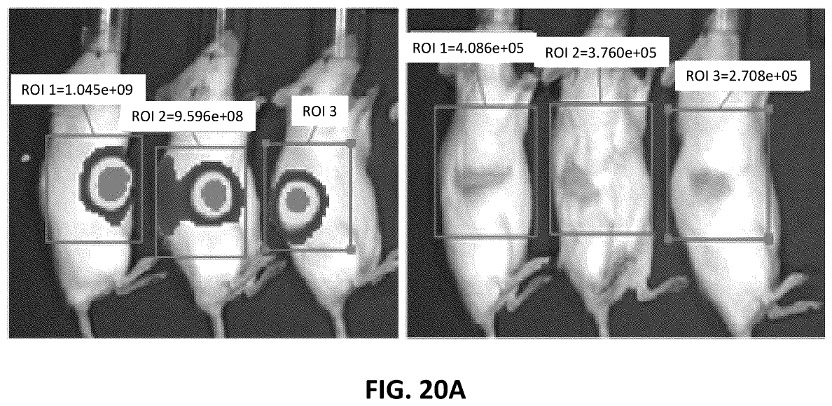

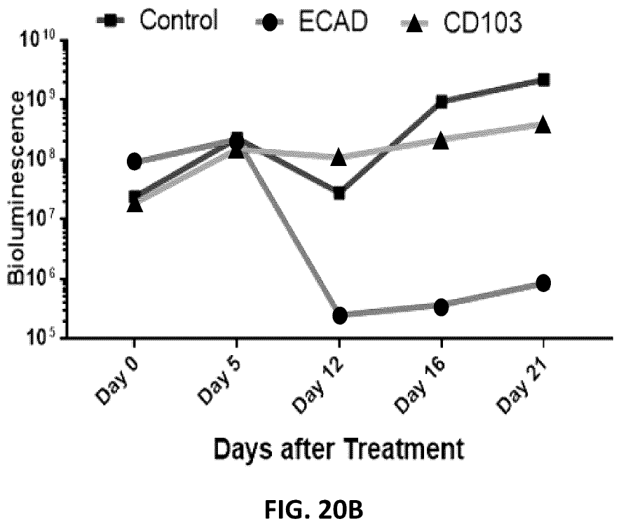

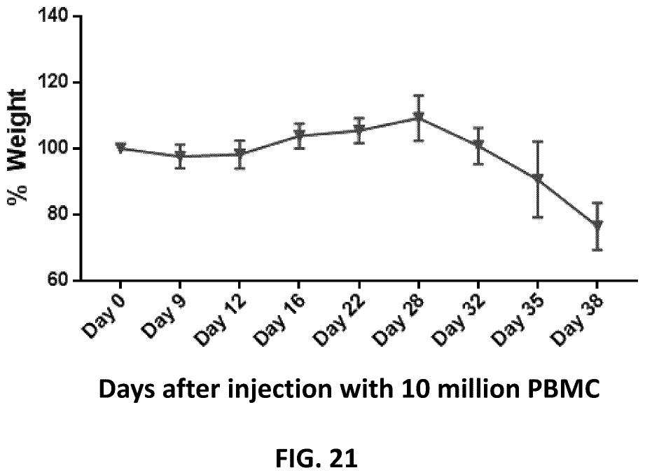

To investigate the ability of CDH1-CAR Tregs to treat aGVHD, humanized mouse models were randomized to receive CDH1-CAR Treg, control Treg, CDH-1-CAR Teff, control Teff, CD103^{high} Tregs, or CD103^{high} Teff (FIG. 5). CDh1-CAR Tregs ameliorated GVHD, and CDh1-CAR-Teff cells exacerbated GVHD. Results are presented in FIGS. 20A-20B and FIG. 21.

### Example 4: Transduced Effector T-Cells with Anti-CD103-CAR

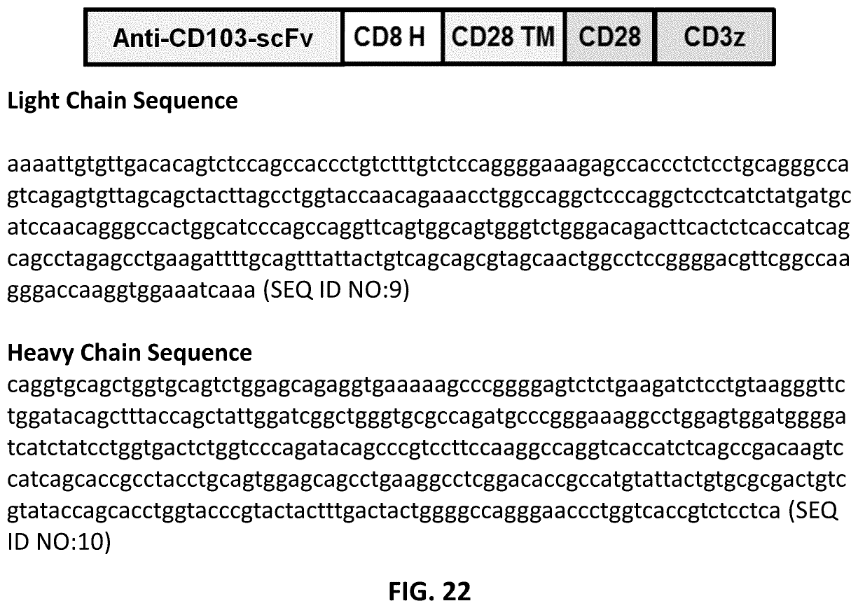

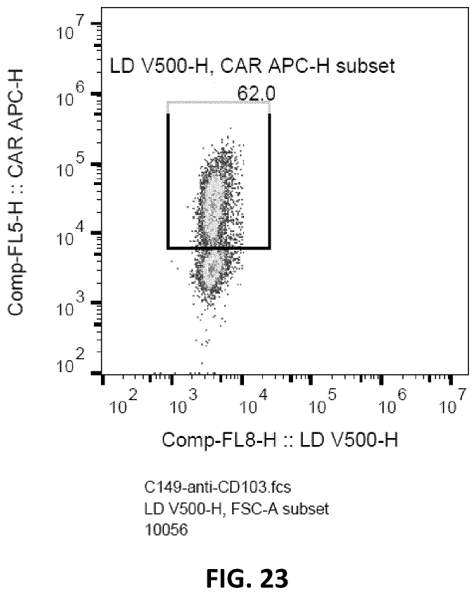

An anti-CD103 CAR construct was generated in which an CD103 scF was built into a second generation CAR with a CD28 co-stimulatory signal (FIG. 22). Using lentivirus technology, effector T-cells were successfully transduced with the anti-CD103-CAR. See, FIG. 23.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a mammal having graft versus host disease (GVHD), wherein said method comprises administering to said mammal a composition comprising regulatory T cells (Tregs) comprising exogenous nucleic acid encoding an antigen receptor targeting an epithelial-specific antigen, wherein said Tregs express said antigen receptor.

2. The method of claim 1, wherein said mammal is a human.

3. The method of claim 1, wherein said GVHD is acute GVHD.

4. The method of claim 1, wherein said GVHD occurred following allogeneic transplantation.

5. The method of claim 1, wherein said epithelial-specific antigen is E-cadherin (CDH1).

6. The method of claim 1, wherein said antigen receptor is a chimeric antigen receptor.

7-9. (canceled)

10. The method of claim 1, wherein said antigen receptor comprises cluster of differentiation 103 (CD103).

11. The method of claim 1, wherein said Tregs, prior to said administration, are engineered to express said antigen receptor ex vivo.

12. (canceled)

13. A method for treating a mammal at risk of developing graft versus host disease (GVHD), wherein said method comprises administering to said mammal a composition comprising regulatory T cells (Tregs) comprising exogenous nucleic acid encoding an antigen receptor targeting an epithelial-specific antigen, wherein said Tregs express said antigen receptor.

14. The method of claim 13, wherein said mammal is a human.

15. The method of claim 13, wherein said GVHD is acute GVHD.

16. The method of claim 13, wherein said mammal received an allogeneic transplantation.

17. The method of claim 13, wherein said epithelial-specific antigen is E-cadherin (CDH1).

18-21. (canceled)

22. The method of claim 13, wherein said antigen receptor comprises cluster of differentiation 103 (CD103).

23. The method of claim 13, wherein said Tregs, prior to said administration, are engineered to express said antigen receptor ex vivo.

24. A nucleic acid construct encoding a chimeric antigen receptor targeting an epithelial-specific antigen.

25. The nucleic acid construct of claim 24, wherein said epithelial-specific antigen is E-cadherin (CDH1).

26-30. (canceled)

31. The nucleic acid construct of claim 24, wherein said chimeric antigen receptor comprises a CD8 hinge region.

32. (canceled)

33. The nucleic acid construct of claim 24, wherein said chimeric antigen receptor comprises a CD28 transmembrane domain.

34. (canceled)

35. The nucleic acid construct of claim 24, wherein said chimeric antigen receptor comprises a CD3zeta signaling domain.

36. (canceled)


SEQUENCE LISTING (10 sequences)

SEQ ID NO: 1 - DNA, 63 bp, Homo sapiens
  source 1..63: mol_type = other DNA
atggccttaccagtgaccgccttgctcctgccgctggccttgctgctccacgccgccaggccg

SEQ ID NO: 2 - DNA, 361 bp, synthetic construct
  source 1..361: mol_type = other DNA
gaggtgcagctggtggagtctgggggaggcttggtacagcctggggggtccctgagactctcctgtgcagcctctggattcaccttcagtagctatggcatgcactgggtccgccaggctccagggaaggggctggagtgggttgcatacattactactagaagtagtaccatatactacgcagactctgtgaagggccgattcaccatctccagagacaatgccaagaactcactgtatctgcaaatgaacagcctgagagccgaggacacggctgtgtattactgtactagagaacccctaactggatactatgctatggactactggggtcaaggaacctcagtcaccgtctcctcag

SEQ ID NO: 3 - DNA, 60 bp, synthetic construct
  source 1..60: mol_type = other DNA
ggaggtggcggatcaggcggaggaggcagcggcggaggtggatcaggaggcggagggtca

SEQ ID NO: 4 - DNA, 336 bp, synthetic construct
  source 1..336: mol_type = other DNA
gatgttgtgatgactcagtctccactctccctgcccgtcacccttggacagccggcctccatctcctgcaggtctagtcaaagcatcgtacacagtgatggaaacacctacttggaatggtatcagcagaggccaggccaatctccaaggcgcctaatttataaggtttctaaccggttctctggggtcccagacagattcagcggcagtgggtcaggcactgatttcacactgaaaatcagcagggtggaggctgaggatgttggggttattactgctttcaaggttcacatgctccgtggacgttcggtggaggcaccaaggtggaaatcaaac

SEQ ID NO: 5 - DNA, 135 bp, Homo sapiens
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 6 - DNA, 72 bp, Homo sapiens
  source 1..72: mol_type = other DNA
atctacatctgggcgcccttggccgggacttgtggggtccttctcctgtcactggttatcaccctttactgc

SEQ ID NO: 7 - DNA, 123 bp, Homo sapiens
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 8 - DNA, 336 bp, synthetic construct
  source 1..336: mol_type = other DNA
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 9 - DNA, 324 bp, synthetic construct
  source 1..324: mol_type = other DNA
aaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccggggacgttcggccaagggaccaaggtggaaatcaaa

SEQ ID NO: 10 - DNA, 369 bp, synthetic construct
  source 1..369: mol_type = other DNA
caggtgcagctggtgcagtctggagcagaggtgaaaaagcccggggagtctctgaagatctcctgtaagggttctggatacagctttaccagctattggatcggctgggtgcgccagatgcccgggaaaggcctggagtggatggggatcatctatcctggtgactctggtcccagatacagcccgtccttccaaggccaggtcaccatctcagccgacaagtccatcagcaccgcctacctgcagtggagcagcctgaaggcctcggacaccgccatgtattactgtgcgcgactgtcgtataccagcacctggtacccgtactactttgactactggggccagggaaccctggtcaccgtctcctca